(19) United States
(12) Patent Application Publication
(10) Pub. No.: US 2016/0114125 A1
(43) Pub. Date: Apr. 28, 2016

(54) Variable Stiffness Balloon Catheter and Related Systems and Methods

(71) Applicant: QXMedical, LLC, Roseville, MN (US)

(72) Inventors: Fernando Di Caprio, St. Paul, MN (US); Gianfranco Panarello, Montreal (CA)

(21) Appl. No.: 14/922,736

(22) Filed: Oct. 26, 2015

Related U.S. Application Data
(60) Provisional application No. 62/068,911, filed on Oct. 27, 2014.

(51) Int. Cl. A61M 25/00; A61B 5/02; A61B 17/12; A61M 25/10; A61F 2/958

(52) U.S. Cl. CPC A61M 25/0054; A61M 25/10; A61F 2/958; A61B 17/12109; A61B 5/02007

(57) ABSTRACT

The various embodiments herein relate to balloon catheters, and more specifically to balloon catheters having variable stiffnesses.

### CROSS-REFERENCE TO RELATED APPLICATIONS

This application claims the benefit under 35 U.S.C. §119(e) to U.S. Provisional Application 62/068,911, filed Oct. 27, 2014 and entitled “Variable Stiffness Balloon Catheter and Related Methods,” which is hereby incorporated herein by reference in its entirety.

### FIELD OF THE INVENTION

The various embodiments disclosed herein relate to balloon catheters for use as medical devices in various procedures, including, but not limited to, endovascular aortic repair, occlusion, and vessel sizing. More specifically, the various embodiments relate to such balloon catheters having variable shaft stiffnesses.

### BACKGROUND OF THE INVENTION

The use of balloon catheters during various procedures, including endovascular aortic repair (“EVAR”) procedures, is well known. In a typical EVAR procedure, one or more covered stents are placed in the aorta or a vessel in the neighboring vasculature, to treat an aneurysm. The covered stent(s) allows the blood to flow through the stent(s) and thus relieves the aneurysm of the blood pressure (thus preventing the aneurysm from rupture). In certain procedures, a non-covered stent or flow diverter is used. The stents are delivered on a catheter inserted through an entry site (typically the femoral artery in the groin area) via an introducer sheath and over a guidewire. Once the stents are deployed, a balloon catheter is inserted over a guidewire through the entry site. The balloon catheter is advanced to the area in the vessel where a stent graft has been deployed and then inflated in order to improve the apposition of the stent graft within the vessel and to mold the stent after placement. The covered stent typically has a fabric within its structure and the molding (or modeling) also helps smooth out any wrinkles. The balloon may also be inflated within the various vessels in order to occlude blood flow as may be required during EVAR or other medical procedures. Other medical procedures may utilize the balloon catheter to size or remodel vessels or other anatomical structures.

A known, single stiffness balloon catheter 10 is shown in FIG. 1. The catheter 10 has a catheter shaft 12, a handle 14 coupled at the proximal end of the shaft 12, an expandable balloon 16 bonded to the shaft 12, two radiopaque markers 18A, 18B, a distal tip 20, and a distal opening 22 defined in the tip 20. The handle 14 has a balloon inflation port 24 that can have a flow-limiting switch 26. This port 24 (along with the flow-limiting switch 26) is in fluid communication with the balloon 16 (via at least one lumen (not shown) in the catheter shaft 12) to allow balloon inflation. A second port 28 on the handle 14 is in fluid communication with a lumen (not shown) along the catheter shaft 12 and the distal opening 22 to allow passage over a guidewire. The ports on the handle 14 (& flow-limiting switch 26) allow connection to standard syringes or inflation devices. All of the aortic modeling, occlusion, or sizing balloon catheters on the market right now have a shaft 12 with a single stiffness along the length of that shaft 12.

There is a need in the art for improved balloon catheters for use in various medical procedures, including EVAR procedures.

### BRIEF SUMMARY OF THE INVENTION

Discussed herein are various balloon catheters having shaft segments and/or zones of varying stiffness and related methods for performing procedures with such catheters.

In Example 1, a balloon catheter comprises a catheter shaft and an expandable balloon operably coupled to the catheter shaft. The shaft comprises a proximal segment of the catheter shaft having a first predetermined stiffness, a distal segment of the catheter shaft having a second predetermined stiffness, and a transition zone disposed between the proximal segment and the distal segment, wherein the transition zone comprises a stiffness that varies gradually along a length of the zone.

Example 2 relates to the balloon catheter according to Example 1, wherein the second predetermined stiffness is lower than the first predetermined stiffness.

Example 3 relates to the balloon catheter according to Example 1, wherein the first and second predetermined stiffnesses are higher than any portion of the stiffness of the transition zone.

Example 4 relates to the balloon catheter according to Example 1, wherein the transition zone comprises proximal, middle and distal portions, wherein the proximal and distal portions are stiffer than the middle portion.

Example 5 relates to the balloon catheter according to Example 1, wherein the transition zone comprises a proximal portion and a distal portion, wherein the proximal portion is stiffer than the distal portion.

Example 6 relates to the balloon catheter according to Example 1, further comprising a second transition zone disposed distally of the distal segment, wherein the second transition zone comprises a proximal portion and a distal portion, wherein the proximal portion is more flexible than the distal portion.

Example 7 relates to the balloon catheter according to Example 6, further comprising a second distal segment distal to the second transition zone, wherein the second distal segment has a predetermined stiffness.

Example 8 relates to the balloon catheter according to Example 1, wherein the gradual variation in the stiffness is caused by a gradual variation in a composition of the transition zone.

Example 9 relates to the balloon catheter according to Example 8, wherein the gradual variation in the composition comprises a gradual variation in amounts of at least two different components.

Example 10 relates to the balloon catheter according to Example 1, wherein a proximal portion of the transition zone comprises a stiffness substantially similar to the first predetermined stiffness and a distal portion of the transition zone comprises a stiffness substantially similar to the second predetermined stiffness.

In Example 11, a method for performing a medical procedure comprises inserting a balloon catheter over a guidewire into a blood vessel through an entry site, positioning the balloon within an anatomical structure, inflating the balloon, deflating the balloon, and retracting the balloon catheter from the blood vessel. The balloon catheter comprises a catheter shaft and an expandable balloon operably coupled to the catheter shaft. The catheter shaft comprises a proximal segment of the catheter shaft having a predetermined stiffness, a distal segment of the catheter shaft having a predetermined stiffness that is lower than the proximal segment, and a transition zone disposed between the proximal segment and the distal segment, wherein the transition zone comprises a stiffness that varies gradually along a length of the zone.

Example 12 relates to the method according to Example 11, wherein the medical procedure is an endovascular aortic repair procedure, wherein the blood vessel is an aorta or neighboring vessel, wherein the inflating the balloon further comprises expanding a stent or improving apposition of the stent.

Example 13 relates to the method according to Example 11, wherein the medical procedure is vessel occlusion.

Example 14 relates to the method according to Example 11, wherein the medical procedure is sizing of the blood vessel or another anatomical structure.

Example 15 relates to the method according to Example 11, wherein the medical procedure is vessel or structural remodeling.

In Example 16, a method for performing a medical procedure comprises inserting a balloon catheter over a guidewire into a blood vessel through an entry site, positioning the balloon within an anatomical structure, inflating the balloon, deflating the balloon, and retracting the balloon catheter from the blood vessel. The balloon catheter comprises a catheter shaft and an expandable balloon operably coupled to the catheter shaft. The catheter shaft comprises a proximal segment of the catheter shaft having a predetermined stiffness, a distal segment of the catheter shaft having a predetermined stiffness that is lower than the proximal length, and a middle segment disposed between the proximal segment and the distal segment.

Example 17 relates to the method according to Example 16, wherein the medical procedure is an endovascular aortic repair procedure, wherein the blood vessel is an aorta or neighboring vessel, wherein the inflating the balloon further comprises expanding a stent or improving apposition of the stent.

Example 18 relates to the method according to Example 16, wherein the medical procedure is vessel occlusion.

Example 19 relates to the method according to Example 16, wherein the medical procedure is sizing of the blood vessel or another anatomical structure.

Example 20 relates to the method according to Example 16, wherein the medical procedure is vessel or structural remodeling.

Example 21 relates to the method according to Example 16, wherein the middle segment has a stiffness that is lower than the proximal segment and higher than the distal segment.

Example 22 relates to the method according to Example 16, wherein the middle segment has a stiffness that is substantially the same as the proximal segment.

Example 23 relates to the method according to Example 16, wherein the middle segment has a stiffness that is substantially the same as the distal segment.

In Example 24, a method for performing a medical procedure comprises inserting a balloon catheter over a guidewire into a blood vessel through an entry site, positioning the balloon within an anatomical structure, inflating the balloon, deflating the balloon, and retracting the balloon catheter from the blood vessel. The balloon catheter comprises a catheter shaft and an expandable balloon operably coupled to the catheter shaft. The catheter shaft comprises a transition zone comprising a stiffness that varies gradually along a length of the zone, wherein a proximal portion of the transition zone is stiffer than a distal portion of the transition zone.

Example 25 relates to the method according to Example 24, wherein the medical procedure is an endovascular aortic repair procedure, wherein the blood vessel is an aorta or neighboring vessel, wherein the inflating the balloon further comprises expanding a stent or improving apposition of the stent.

Example 26 relates to the method according to Example 24, wherein the medical procedure is vessel occlusion.

Example 27 relates to the method according to Example 24, wherein the medical procedure is sizing of the blood vessel or another anatomical structure.

Example 28 relates to the method according to Example 24, wherein the medical procedure is vessel or structural remodeling.

Example 29 relates to the method according to Example 24, wherein the first predetermined stiffness is a higher stiffness, wherein the catheter shaft further comprises a higher stiffness segment distal to the transition zone.

Example 30 relates to the method according to Example 24, wherein the catheter shaft further comprises a proximal segment of the catheter shaft having a first predetermined stiffness, wherein the proximal segment is disposed proximally of the transition zone.

Example 31 relates to the method according to Example 24, wherein the catheter shaft further comprises a distal segment of the catheter shaft having a second predetermined stiffness, wherein the distal segment is disposed distally of the transition zone.

In Example 32, a method for performing a medical procedure comprises inserting a balloon catheter over a guidewire through an entry site, positioning the balloon within an anatomical structure, inflating, deflating the balloon, and retracting the balloon catheter from the blood vessel. The balloon catheter comprises a catheter shaft and an expandable balloon operably coupled to the catheter shaft. The catheter shaft comprises first higher stiffness length at a proximal portion of the catheter shaft, a first medium stiffness length distal to the first higher stiffness length, a higher flexibility length distal to the first medium stiffness length, and a second medium stiffness length distal to the higher flexibility length.

Example 33 relates to the method according to Example 32, wherein the medical procedure is an endovascular aortic repair procedure, wherein the blood vessel is an aorta or neighboring vessel, wherein the inflating the balloon further comprises expanding a stent or improving apposition of the stent.

Example 34 relates to the method according to Example 32, wherein the medical procedure is vessel occlusion.

Example 35 relates to the method according to Example 32, wherein the medical procedure is sizing of the blood vessel or another anatomical structure.

Example 36 relates to the method according to Example 32, wherein the medical procedure is vessel or structural remodeling.

Example 37 relates to the method according to Example 32, wherein the catheter shaft further comprises a second higher stiffness length distal to the second medium stiffness length.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a side view of a known, single stiffness balloon catheter.

FIG. 2 is a side view of a variable stiffness balloon catheter having a higher stiffness length, a lower stiffness length, and a transition zone, according to one embodiment.

FIG. 3 is a side view of a variable stiffness balloon catheter having a higher stiffness segment, a medium stiffness segment, and a lower stiffness segment, according to another embodiment.

FIG. 4 is a side view of a variable stiffness balloon catheter having a higher stiffness length and an extended transition zone, according to another embodiment.

FIG. 5 is a side view of a variable stiffness balloon catheter having a shaft with multiple segments of varying stiffnesses, according to another embodiment.

FIG. 6 is a side view of a variable stiffness balloon catheter having a first higher stiffness length, a transition zone, and a second higher stiffness length, according to a further embodiment.

FIG. 7 is a side view of a variable stiffness balloon catheter having a first higher stiffness length, a first transition zone with a higher stiffness at the proximal end of the zone that changes gradually along its length to a lower stiffness at or near the distal end, a higher flexibility length, and a second transition zone with a lower stiffness at the proximal end of the zone that changes gradually along its length to a higher stiffness at or near the distal end, according to yet another embodiment.

FIG. 8A is a cross-sectional view of a shaft of a balloon catheter, according to one implementation.

FIG. 8B is a cross-sectional view of a shaft of a balloon catheter, according to another implementation.

FIG. 8C is a cross-sectional view of a shaft of a balloon catheter, according to a further implementation.

FIG. 8D is a cross-sectional view of a shaft of a balloon catheter, according to yet another implementation.

FIG. 8E is a cross-sectional view of a shaft of a balloon catheter, according to another embodiment.

FIG. 8F is a cross-sectional view of a shaft of a balloon catheter, according to a further embodiment.

FIG. 9 is a perspective and partial cross-sectional view of a balloon catheter with a multi-lumen shaft, according to one embodiment.

FIG. 10 is a perspective and partial cross-sectional view of a balloon catheter with a coaxial shaft, according to one embodiment.

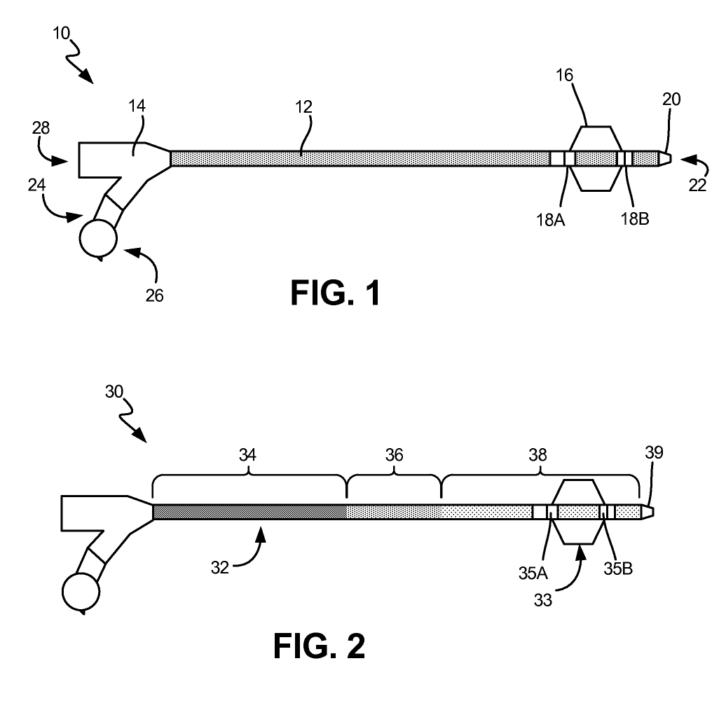

### DETAILED DESCRIPTION

The various embodiments disclosed herein relate to balloon catheters for use in various medical procedures, including EVAR procedures.

With the advent of increasingly more flexible stent grafts, more stent grafts are being implanted into tortuous and highly angulated blood vessels, including, for example, the aorta or neighboring vessels. This creates a need for balloon catheters that have more distal flexibility and trackability. That is, it creates a need for balloon catheters that are more flexible, at least on the distal end, in order to access the deployed stents and more trackable to navigate more tortuous vasculature. It is still advantageous to have a less flexible proximal section in order to maintain good catheter pushability.

The implementations disclosed and contemplated herein relate to variable stiffness balloon catheters, including variable stiffness multi-lumen and coaxial catheters, for use in various medical procedures, including EVAR procedures. In certain balloon catheter embodiments, the shaft has a stiffer proximal end and a relatively more flexible distal end. This gives the catheter embodiments the characteristics of having similar pushability features in the proximal portion as known balloon catheters while having a distal end that is more flexible and trackable to conform and track through more tortuous or angulated vessels and anatomical structures.

It is understood that the term “stiffness” as used herein means bending stiffness of a catheter shaft, which is the resistance of a beam-like member (such as a catheter shaft) to lateral deformation or deflection given the application of a lateral force. In certain implementations as described herein, a material with a higher durometer is described as a material that has a higher stiffness than a material with a lower durometer. In this context, it is understood that a catheter shaft made of a higher durometer material has a higher stiffness than catheter shaft of the same geometry and cross-sectional area made of a lower durometer material.

One embodiment, as shown in FIG. 2, is a variable stiffness balloon catheter 30 with a shaft 32, a balloon 33, and a distal tip 39. The shaft 32 has a higher stiffness length 34, a lower stiffness (or higher flexibility) length 38, and an extended transition zone 36 in which the stiffness “transitions” or changes gradually along the length of the zone from a higher stiffness at or near the proximal end to a lower stiffness (or greater flexibility) at or near the distal end. In those implementations in which the transition zone has a stiffness that changes gradually along the length of the zone, the transition zone can also be referred to as a “gradual transition zone.” In certain alternative embodiments, the stiffness of the transition zone does not change gradually. In the embodiment of FIG. 2, the transition zone 36 constitutes less than 25% of the length of the catheter. In another embodiment, the transition zone 36 extends along the entire length of the catheter. Alternatively, the transition zone 36 extends along substantially the entire length of the catheter. In a further alternative, the transition zone 36 extends along more than 50% of the length of the catheter (such as FIG. 4 discussed below). In yet another alternative, the transition zone 36 extends along less than 50% of the length of the catheter. Also shown in FIG. 2 are two markers 35A, 35B, according to one embodiment. In this specific example, the markers 35A, 35B are radiopaque markerbands 35A, 35B. Alternatively, the markers 35A, 35B and any other markers disclosed or contemplated herein can take any form or configuration. According to various embodiments, any number of markers can be used. The markers may be placed on the shaft within the balloon component, on the shaft outside the balloon component, or in any other known location for positioning markers on a catheter.

In one embodiment, the transition zone 36 of FIG. 2 (and any transition zone as disclosed or contemplated herein) has a length ranging from about 0.25 inches to the full length of the catheter shaft (in one exemplary embodiment, the catheter shaft can have a length of at least 80 inches). Alternatively, the length of the transition zone ranges from about 0.5 inches to about 15 inches. In a further implementation, the transition ranges from about 2 inches to about 5 inches.

It is understood that the variation in stiffness can be comprised of any known variation. For example, in certain implementations, the transition zone can have a proximal portion that has a first predetermined stiffness, and then there is a transition moving distally to a higher stiffness, followed by a further transition moving further distally to a lower stiffness. In a different alternative embodiment, the transition zone can have a proximal portion that has a first predetermined stiffness, and then there is a transition moving distally to a lower stiffness, followed by a further transition moving further distally to a higher stiffness. In yet another alternative, the transition zone can have a variation in stiffness that changes linearly along at least a part of the transition zone.

In accordance with certain implementations, the variation in the stiffness of the transition zone is accomplished by predetermined variation in the amounts of at least two different materials that make up the transition zone. Alternatively, the variation can be accomplished using predetermined variation in the amounts of three or more different materials.

For example, in one embodiment, a variable stiffness catheter with a transition zone is produced during the shaft extrusion process. That is, a two-way valve mechanism on the extruders allows the extruded material to switch between a high stiffness material (such as a 72 durometer (“D”) or 63D PEBA-based compound) and a low stiffness material (such as a 35D or 40D PEBA-based compound or polyurethane). In one specific example, the extruded material transitions from a 72D PEBA-based compound to a 63D PEBA-based compound. In another specific example, the extruded material transitions from a 72D PEBA-based compound to a 55D PEBA-based compound. It is understood that alternative implementations have transitions from any known higher stiffness material to any known lower stiffness material. The switch from the high stiffness material to the low stiffness material creates the transition zone (such as the transition zone 36 discussed above and depicted in FIG. 2).

Alternately, the two-way valve on the extruder may also be set up in a manner that allows both high & low stiffness materials to be extruded simultaneously in varying amounts. For example, the extrusion may have 90% of the high stiffness material & 10% of the low stiffness material being extruded and then transition to have 10% of the high stiffness material & 90% of the low stiffness material being extruded. Alternatively, the transition zone (whether extruded or produced by any other known process) can be made up of any combination of two materials: one of high stiffness and one of low stiffness. For example, the amount of the two materials can vary from 99% of the high stiffness material (and 1% of the low stiffness material) to 1% of the high stiffness material (and 99% of the low stiffness material). Further, any amount in between can also be used. For example, some portion of the transition zone can have 80% high stiffness material and 20% low stiffness material. Alternatively, some portion of the transition zone can have 80% low stiffness material and 20% high stiffness material. In a further implementation, some portion of the transition zone can have 70% high stiffness material and 30% low stiffness material. Alternatively, some portion of the transition zone can have 70% low stiffness material and 30% high stiffness material. It is understood that any other combination is also possible. It is further understood that the transition zone in other alternative embodiments can be made up of three or more different materials of different stiffnesses that can vary in any known amounts.

The various embodiments herein can be constructed using an extrusion process such as that described above. Alternatively, any other known process for constructing catheters of variable stiffness as described herein can be used to create the implementations described herein. Further, any known materials or components of varying stiffnesses can be used to create the catheter shafts as described herein.

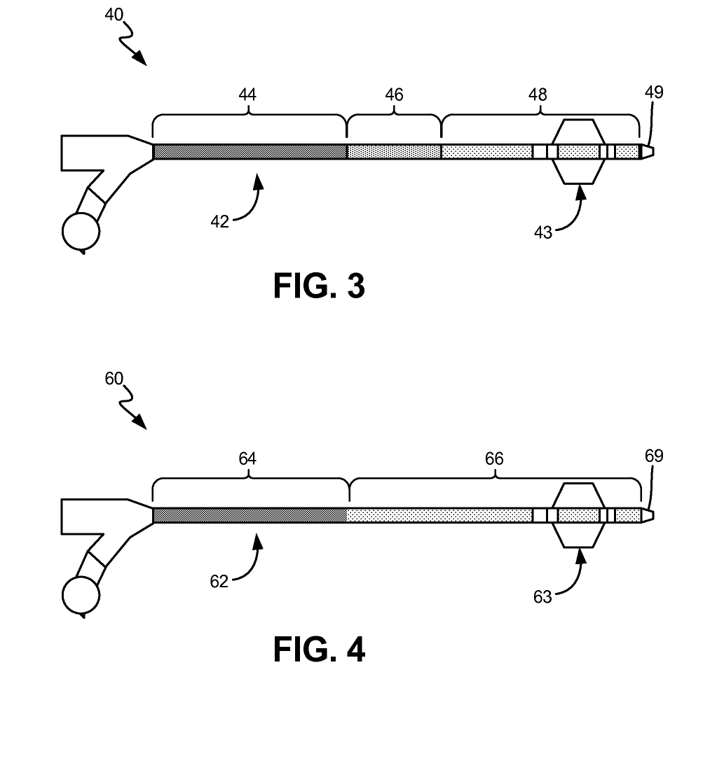

FIG. 3 depicts another embodiment of a variable stiffness balloon catheter 40 with a shaft 42, a balloon 43, and a distal tip 49. The shaft 42 has discrete segments that are joined together. More specifically, the shaft 42 has a higher stiffness segment 44, a medium stiffness segment 46, and a lower stiffness segment 48. Each of the segments 44, 46, 48 are bonded, welded, or otherwise attached to each other to produce the full shaft 42. Each of the segments 44, 46, 48 is made of a different material to reflect the desired amount of stiffness. Alternatively, the shaft 42 can have only two (rather than three) different stiffness segments—a higher stiffness segment and a lower stiffness segment. In a further alternative, the shaft 42 can have more than 3 segments, each of different flexibility.

FIG. 4, according to one implementation, is a variable stiffness balloon catheter 60 with a shaft 62, a balloon 63, and a distal tip 69. The shaft 62 has a higher stiffness length 64 and an extended transition zone 66 in which the stiffness “transitions” or changes gradually along the length of the zone 66 from a higher stiffness at or near the proximal end of the length to a lower stiffness (or greater flexibility) at or near the distal end. In this embodiment, the transition zone 66 extends along more than 50% of the length of the catheter. Alternatively, the transition zone 66 can constitutes less than 50% of the length of the catheter.

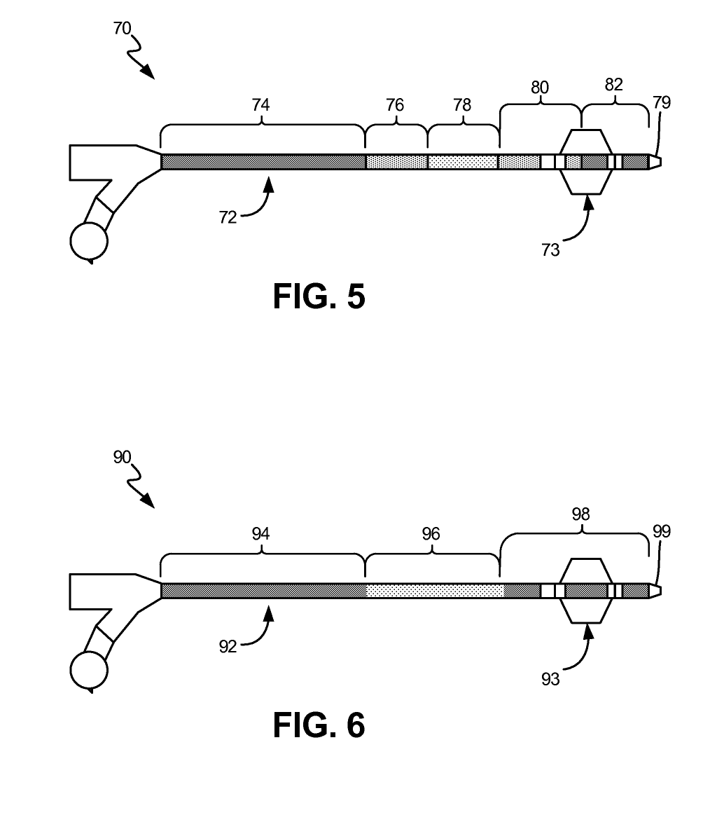

FIG. 5 depicts a further embodiment in which the variable stiffness balloon catheter 70 has a shaft 72 with multiple stiffnesses, a balloon 73, and a distal tip 79. More specifically, the shaft 72 has a first higher stiffness length (or segment) 74, a first medium flexibility length (or segment) 76, a higher flexibility length (or segment) 78, a second medium flexibility length (or segment) 80, and a second higher stiffness length (or segment) 82. With respect to FIG. 5 and all other embodiments and figures discussed or contemplated herein, it is understood that each of the lengths or segments can be of any length and further can be positioned anywhere along the entire length of the shaft 72 in comparison to the other lengths or segments.

In a further embodiment, FIG. 6 shows a variable stiffness balloon catheter 90 having a shaft 92, a balloon 93, and a distal tip 99. The shaft has a first higher stiffness length (or segment) 94, a transition zone 96, and a second higher stiffness length (or segment) 98. In the transition zone 96, the stiffness changes gradually along the length of the zone 96 from a higher stiffness at or near the proximal end of the zone to a lower stiffness (or greater flexibility) at or near the middle of the zone 96 and further to a higher stiffness at or near the distal end of the zone 96. Alternatively, the zone 96 can have a length or portion having a constant stiffness.

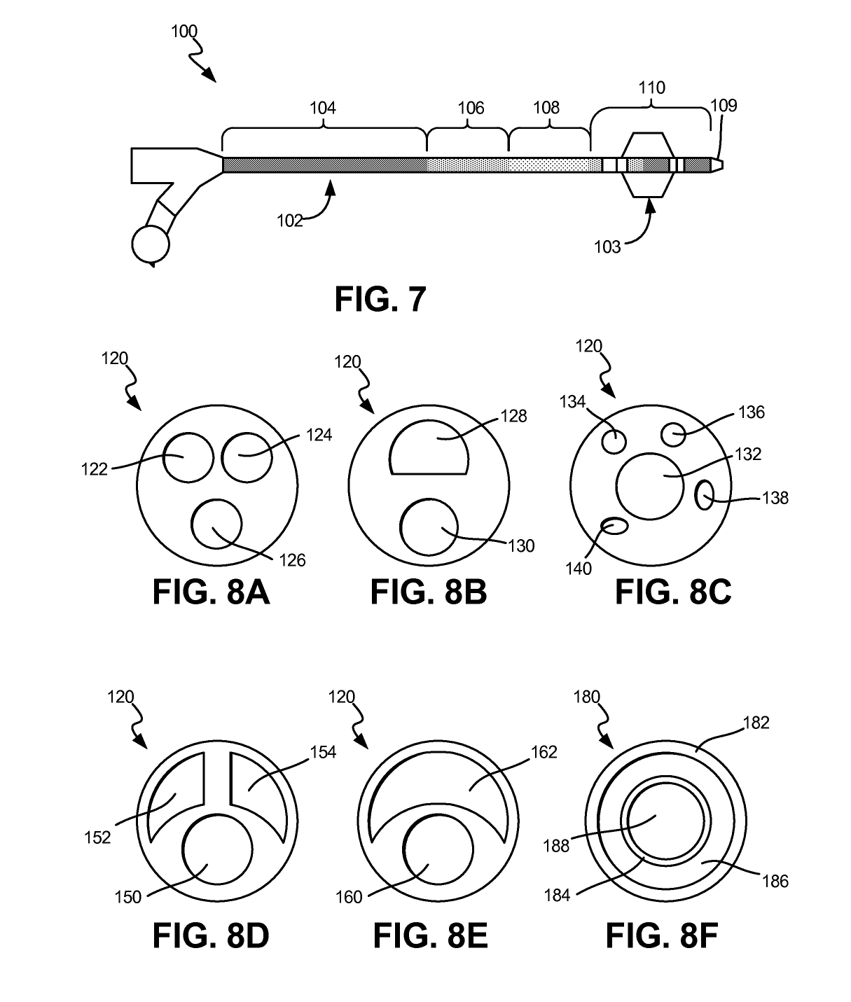

As shown in FIG. 7, another embodiment relates to a variable stiffness balloon catheter 100 having a shaft 102, a balloon 103, and a distal tip 109. The shaft 102 has a first higher stiffness length (or segment) 104, a first transition zone 106 with a higher stiffness at the proximal end of the zone 106 that changes gradually along its length to a lower stiffness at or near the distal end, a higher flexibility length (or segment) 108, and a second transition zone 110 with a lower stiffness at the proximal end of the zone 110 that changes gradually along its length to a higher stiffness at or near the distal end.

Alternative catheter embodiments, and any of the implementations disclosed or contemplated herein, can have any number of zones, lengths, or segments. That is, the number of zones, lengths, or segments can vary from 2 to as many such zones, lengths, or segments as desired or necessary for any such catheter. Further, additional alternative implementations can have zones, lengths, or segments of any length as desired or necessary for any such catheter. It is further understood that any of the zones, lengths, or segments can extend to any portion of any catheter embodiment herein.

According to various additional variable stiffness balloon catheter embodiments, any catheter implementation disclosed (including any embodiment as shown in FIGS. 2-7) or contemplated herein can also have one or more additional characteristics as follows. In one example, any catheter embodiment can have a balloon component (similar to the balloon components 33, 43, 63, 73, 93, or 103 as discussed above with respect to FIGS. 2-7) that is compliant or semi-compliant with a large expansion range. For example, the balloon according to any of the implementations herein can be capable of expanding from about 5 mm to about 60 mm. Alternatively, the balloon can expand from about 5 mm to about 54 mm or from about 5 mm to about 28 mm. In a further alternative, the balloon can expand from about 10 mm to about 60 mm. In yet another alternative, the balloon can expand from about 10 mm to about 54 mm or from about 10 mm to about 28 mm. Alternatively, any embodiment disclosed or contemplated herein can have a substantially non-compliant balloon with a small expansion range.

According to another exemplary implementation, the various catheter implementations can be compatible with guidewires having a diameter ranging from about 0.014 inches to about 0.038 inches. Alternatively, the various implementations are compatible with a guidewire having a diameter ranging from about 0.035 inches to about 0.038 inches. Further known diameters are also compatible, according to further alternative embodiments.

Any catheter embodiment disclosed or contemplated herein can be compatible with introducer sheaths sized 6 Fr or larger. Alternatively, the embodiments herein can be compatible with any sheath sized 8 Fr or larger. In yet another alternative, the embodiments herein can be compatible with any sheath sized 10 Fr or larger.

In accordance with a further implementation, the various balloon catheter embodiments disclosed and contemplated herein can have a distal tip (such as any exemplary distal tip 39, 49, 69, 79, 99, 109 as discussed herein) made with material softer than that of the distal shaft. For example, in one specific embodiment, the tip can be made of a lower durometer material such as 25 or 35 durometer PEBAX material. Alternatively, the tip can be made of polyurethane. In yet another alternative, the tip can be made of any other known material softer than that of the distal shaft.

Certain embodiments of the catheter disclosed and contemplated herein can have an effective length of between about 40 cm and 150 cm. Alternatively, any of the catheter embodiments can have an effective length ranging from about 55 cm to about 110 cm. In a further alternative, any catheter embodiment can have any known length for a balloon catheter.

The balloon catheter, in certain implementations, can have a flow limiting switch (or controller) on the inlet to the balloon inflation port to control inflation or deflation of the balloon.

According to a further embodiment, any balloon disclosed or contemplated herein can be capable of occluding vessels having diameters ranging from about 2 mm to about 60 mm. Alternatively, the occluding vessels can have diameters ranging from about 25 mm to about 45 mm.

As mentioned above, in certain embodiments, the balloon catheter embodiments disclosed or contemplated herein can be also be used for other procedures beyond EVAR procedures. For example, in some implementations, the various catheter embodiments herein can be used for procedures relating to the sizing of vessels or other anatomical structures. Such a procedure can involve inflating the balloon with a radiopaque solution (or other known detectable solution) under fluoroscopy (or other type of detection system) with a system or technology that allows for measurements to be taken. According to another example, in other embodiments, the various catheter embodiments herein—including those with a substantially non-compliant balloon—can be used for angioplasty or stent-deployment procedures. Alternatively, the various catheter embodiments can be used for any known procedure that utilizes a balloon catheter.

In addition, the shaft of any device embodiment disclosed or contemplated herein (such as shaft 32 of FIG. 2 or shaft 42 of FIG. 3) can have two or more lumens defined therein. For example, as shown in cross-section in FIG. 8A, a shaft 120 can have three lumens 122, 124, 126. Alternatively, as shown in FIG. 8B, the shaft 120 can have two lumens 128, 130, with one lumen 128 being larger (and non-circular) in comparison to the other lumen 130. Further, as shown in FIG. 8C, the shaft 120 can have one large lumen 132 and four smaller lumens 134, 136, 138, 140. Another embodiment is depicted in FIG. 8D, which depicts a shaft 120 having three lumens 150, 152, 154, with lumen 150 being circular and larger and the other two lumens 152, 154 being non-circular. A further embodiment is shown in FIG. 8E, which depicts a shaft 120 having two lumens 160, 162, with lumen 160 being circular and lumen 162 being non-circular. In other implementations, the shaft 120 can have any number of lumens of a variety of sizes and shapes as needed. In each of these embodiments, one of the lumens (typically one of the larger and/or circular lumens) is intended to receive a guidewire, such that the guidewire can be positioned through that lumen (or the device is advanced or retracted over the guidewire via the lumen. Further, one or more of the other lumens can be used for balloon inflation. In a further implementation, one or more of the additional lumens could be used for passage or transmission of one or more fluids (such as contrast agents, diagnostic agents, or saline, for example). That is, the fluid could be injected or otherwise positioned in the lumen at the proximal end of the device and advanced to an exit site at the tip or some point along the length of the shaft (such as shaft 32 or 42).

In another implementation, any shaft of any catheter embodiment disclosed herein (including the shafts 32, 42, 62, 72, 92, 102 disclosed and discussed herein) can be a coaxial shaft, rather than a multi-lumen shaft. One example of such a coaxial shaft 180 is depicted in cross-section in FIG. 8F, with the shaft 180 having an outer conduit 182 defining an outer lumen 186 and an inner conduit 184 defining an inner lumen 188. The inner conduit 184 is disposed within the outer lumen 186 of the outer conduit 182 as shown. In one embodiment, the inner lumen 188 can be configured to receive a guidewire, while the outer lumen 186 between the inner conduit 184 and the outer conduit 182 is configured to receive inflation fluid. Alternatively, the lumens 186, 188 can be configured to receive any known device or function in any known fashion for any known coaxial shaft. It is further understood that any of the variable stiffness embodiments disclosed or contemplated herein can apply to one or both of the inner and outer conduits of a coaxial shaft, and also that different variable stiffness configurations can be incorporated into the conduits such that each conduit can have different stiffness characteristics.

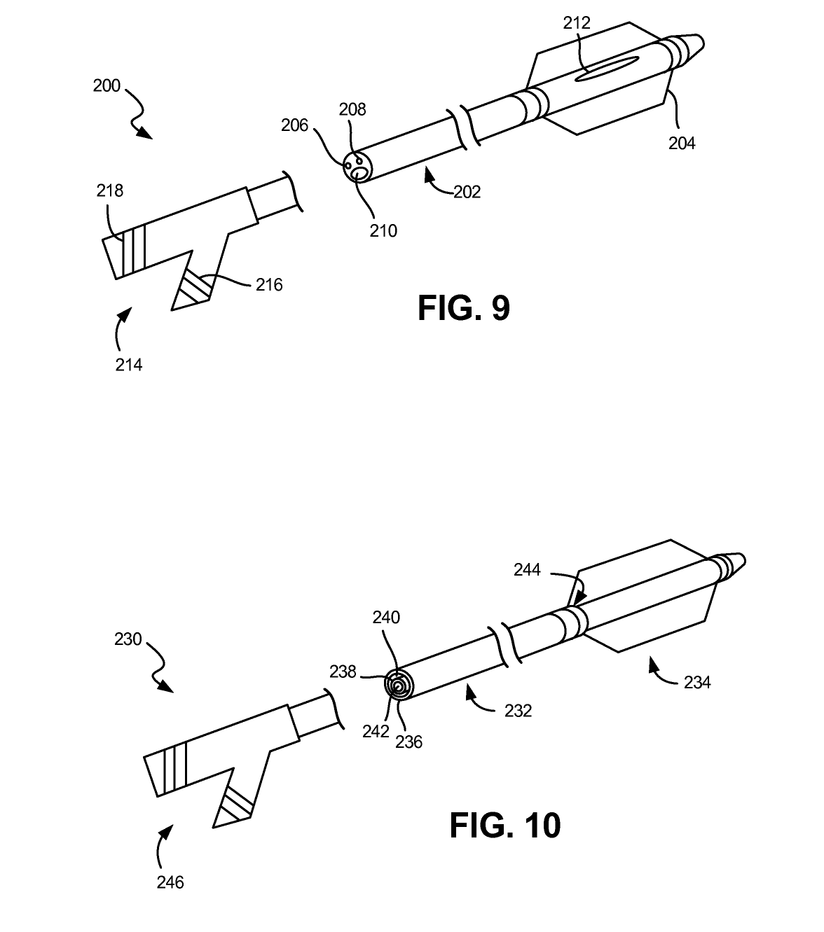

FIG. 9, according to one embodiment, depicts a variable stiffness balloon catheter 200 having a multi-lumen shaft 202, a balloon 204, and three lumens 206, 208, 210. In one implementation as shown, the two lumens 206, 208 are smaller lumens and the lumen 210 is a larger lumen. The lumens 206, 208 in accordance with one embodiment are the inflation lumens that are configured to receive the inflation fluid while the lumen 210 is the guidewire lumen that is configured to receive the guidewire. The catheter 200 also has an opening 212 defined in the shaft 202 in the portion of the shaft 202 that is disposed within the balloon 204 such that the interior of the balloon 204 is in fluid communication with the inflation lumens 206, 208. The catheter 200 also has a handle 214 disposed at a proximal end of the catheter 200 that has an inflation port 216 and a guidewire port 218. Alternatively, the handle 214 can have any known configuration. It is understood that any of the variable stiffness configurations of any of the catheter embodiments discussed above can be incorporated into this catheter 200.

FIG. 10 depicts a variable stiffness balloon catheter 230 having a coaxial shaft 232 and a balloon 234. The coaxial shaft 232 has an outer conduit 236 and an inner conduit 238, with the outer conduit 236 defining an outer lumen 240 in which the inner conduit 238 is disposed. The inner conduit 238 defines an inner lumen 242. In this embodiment, the distal portion of the shaft 232 has an opening 244 that is disposed within the balloon 234 such that the balloon 234 is in fluid communication with the portion of the outer lumen 240 between the inner conduit 238 and the outer conduit 236. Thus, any inflation fluid that passes through the outer lumen 240 (between the inner conduit 238 and outer conduit 236) is in fluid communication with the interior of the balloon 234. The inner lumen 242 is configured to receive a guide wire. Alternatively, the inner lumen 242 and outer lumen 240 can be configured to operate in any known fashion for a balloon catheter. In addition, the catheter 230 has a handle 246, which can be any known handle for use with catheters. It is understood that any of the variable stiffness configurations of any of the catheter embodiments discussed above can be incorporated into this catheter 230. Further, it is also understood that different variable stiffness configurations of any embodiments discussed above can be incorporated into the conduits 236, 238 such that each conduit 236, 238 has a different configuration.

Although the present invention has been described with reference to preferred embodiments, persons skilled in the art will recognize that changes may be made in form and detail without departing from the spirit and scope of the invention.

What is claimed is:

1. A balloon catheter comprising:
(a) a catheter shaft comprising:
(i) a proximal segment of the catheter shaft having a first predetermined stiffness;
(ii) a distal segment of the catheter shaft having a second predetermined stiffness; and
(iii) a transition zone disposed between the proximal segment and the distal segment, wherein the transition zone comprises a stiffness that varies gradually along a length of the zone; and
(b) an expandable balloon operably coupled to the catheter shaft;

2. The balloon catheter of claim 1, wherein the second predetermined stiffness is lower than the first predetermined stiffness.

3. The balloon catheter of claim 1, wherein the first and second predetermined stiffnesses are higher than any portion of the stiffness of the transition zone.

4. The balloon catheter of claim 1, wherein the transition zone comprises proximal, middle and distal portions, wherein the proximal and distal portions are stiffer than the middle portion.

5. The balloon catheter of claim 1, wherein the transition zone comprises a proximal portion and a distal portion, wherein the proximal portion is stiffer than the distal portion.

6. The balloon catheter of claim 1, further comprising a second transition zone disposed distally of the distal segment, wherein the second transition zone comprises a proximal portion and a distal portion, wherein the proximal portion is more flexible than the distal portion.

7. The balloon catheter of claim 6, further comprising a second distal segment distal to the second transition zone, wherein the second distal segment has a predetermined stiffness.

8. The balloon catheter of claim 1, wherein the gradual variation in the stiffness is caused by a gradual variation in a composition of the transition zone.

9. The balloon catheter of claim 8, wherein the gradual variation in the composition comprises a gradual variation in amounts of at least two different components.

10. The balloon catheter of claim 1, wherein a proximal portion of the transition zone comprises a stiffness substantially similar to the first predetermined stiffness and a distal portion of the transition zone comprises a stiffness substantially similar to the second predetermined stiffness.

11. A method for performing a medical procedure, the method comprising:
inserting a balloon catheter over a guidewire into a blood vessel through an entry site, the balloon catheter comprising:
(a) a catheter shaft comprising:
(i) a proximal segment of the catheter shaft having a predetermined stiffness;
(ii) a distal segment of the catheter shaft having a predetermined stiffness that is lower than the proximal segment; and
(iii) a transition zone disposed between the proximal segment and the distal segment, wherein the transition zone comprises a stiffness that varies gradually along a length of the zone; and
(b) an expandable balloon operably coupled to the catheter shaft;
positioning the balloon within an anatomical structure;
inflating the balloon;
deflating the balloon; and
retracting the balloon catheter from the blood vessel.

12. The method of claim 11, wherein the medical procedure is an endovascular aortic repair procedure, wherein the blood vessel is an aorta or neighboring vessel, wherein the inflating the balloon further comprises expanding a stent or improving apposition of the stent.

13. The method of claim 11, wherein the medical procedure is vessel occlusion.

14. The method of claim 11, wherein the medical procedure is sizing of the blood vessel or another anatomical structure.

15. The method of claim 11, wherein the medical procedure is vessel or structural remodeling.

16. A method for performing a medical procedure, the method comprising:
inserting a balloon catheter over a guidewire into a blood vessel through an entry site, the balloon catheter comprising:
(a) a catheter shaft comprising:
(i) a proximal segment of the catheter shaft having a predetermined stiffness;
(ii) a distal segment of the catheter shaft having a predetermined stiffness that is lower than the proximal length; and
(iii) a middle segment disposed between the proximal segment and the distal segment; and
(b) an expandable balloon operably coupled to the catheter shaft;
positioning the balloon within an anatomical structure;
inflating the balloon;
deflating the balloon; and
retracting the balloon catheter from the blood vessel.

17. The method of claim 16, wherein the medical procedure is an endovascular aortic repair procedure, wherein the blood vessel is an aorta or neighboring vessel, wherein the inflating the balloon further comprises expanding a stent or improving apposition of the stent.

18. The method of claim 16, wherein the medical procedure is vessel occlusion.

19. The method of claim 16, wherein the medical procedure is sizing of the blood vessel or another anatomical structure.

20. The method of claim 16, wherein the medical procedure is vessel or structural remodeling.

21. The method of claim 16, wherein the middle segment has a stiffness that is lower than the proximal segment and higher than the distal segment.

22. The method of claim 16, wherein the middle segment has a stiffness that is substantially the same as the proximal segment.

23. The method of claim 16, wherein the middle segment has a stiffness that is substantially the same as the distal segment.

24. A method for performing a medical procedure, the method comprising:
inserting a balloon catheter over a guidewire into a blood vessel through an entry site, the balloon catheter comprising:
(a) a catheter shaft comprising
a transition zone comprising a stiffness that varies gradually along a length of the zone, wherein a proximal portion of the transition zone is stiffer than a distal portion of the transition zone; and
(b) an expandable balloon operably coupled to the catheter shaft;
positioning the balloon within an anatomical structure;
inflating the balloon;
deflating the balloon; and
retracting the balloon catheter from the blood vessel.

25. The method of claim 24, wherein the medical procedure is an endovascular aortic repair procedure, wherein the blood vessel is an aorta or neighboring vessel, wherein the inflating the balloon further comprises expanding a stent or improving apposition of the stent.

26. The method of claim 24, wherein the medical procedure is vessel occlusion.

27. The method of claim 24, wherein the medical procedure is sizing of the blood vessel or another anatomical structure.

28. The method of claim 24, wherein the medical procedure is vessel or structural remodeling.

29. The method of claim 24, wherein the first predetermined stiffness is a higher stiffness, wherein the catheter shaft further comprises a higher stiffness segment distal to the transition zone.

30. The method of claim 24, wherein the catheter shaft further comprises a proximal segment of the catheter shaft having a first predetermined stiffness, wherein the proximal segment is disposed proximally of the transition zone.

31. The method of claim 24, wherein the catheter shaft further comprises a distal segment of the catheter shaft having a second predetermined stiffness, wherein the distal segment is disposed distally of the transition zone.

32. A method for performing a medical procedure, the method comprising:
inserting a balloon catheter over a guidewire through an entry site, the balloon catheter comprising:
(a) a catheter shaft comprising:
(i) first higher stiffness length at a proximal portion of the catheter shaft;
(ii) a first medium stiffness length distal to the first higher stiffness length;
(iii) a higher flexibility length distal to the first medium stiffness length; and
(iv) a second medium stiffness length distal to the higher flexibility length; and
(b) an expandable balloon operably coupled to the catheter shaft;
positioning the balloon within an anatomical structure;
inflating;
deflating the balloon; and
retracting the balloon catheter from the blood vessel.

33. The method of claim 32, wherein the medical procedure is an endovascular aortic repair procedure, wherein the blood vessel is an aorta or neighboring vessel, wherein the inflating the balloon further comprises expanding a stent or improving apposition of the stent.

34. The method of claim 32, wherein the medical procedure is vessel occlusion.

35. The method of claim 32, wherein the medical procedure is sizing of the blood vessel or another anatomical structure.

36. The method of claim 32, wherein the medical procedure is vessel or structural remodeling.

37. The method of claim 32, wherein the catheter shaft further comprises a second higher stiffness length distal to the second medium stiffness length.
